(19) United States
(12) Patent Application Publication
(10) Pub. No.: US 2025/0242168 A1
(43) Pub. Date: Jul. 31, 2025

(54) Light Based Therapy Mouthpiece

(71) Applicant: Designs for Vision, Inc., Bohemia, NY (US)

(72) Inventor: Richard E. Feinbloom, New York, NY (US)

(21) Appl. No.: 18/640,888

(22) PCT Filed: Jul. 20, 2023
(86) PCT No.: PCT/US2023/019274

§ 371 Date: Apr. 19, 2024

Related U.S. Application Data
(63) Continuation-in-part of application No. PCT/US2022/049067, filed on Nov. 6, 2022.

(51) Int. Cl. A61N 5/06; A61N 5/00

(52) U.S. Cl. CPC A61N 5/0603; A61N 2005/005; A61N 2005/0606; A61N 2005/0629; A61N 2005/0632; A61N 2005/0667

(57) ABSTRACT

A light based therapy device for providing light-therapy to an oral cavity of a patient, wherein the light based therapy devices comprises a mouth piece element including a plurality of lighting sources configured to emit light into the oral cavity, a control module configured to selectively provide electrical energy to the light sources to cause the lighting sources to emit light wherein, the control module provides the electrical energy to the lighting sources when the indication of proper placement is received. In one aspect, an active cooling device is incorporated to maintain substantially consistent temperature of the device within the body cavity.

### CLAIM OF PRIORITY

This applicant application is a U. S. National Phase application under 35 U.S.C. 371 of the International Application No. PCT/US2023/019274 filed on Apr. 20, 2023, which published as WO (not yet published), which claims, pursuant to 35 USC 120, priority to, and the benefit of the earlier filing date of that patent application filed on Nov. 6, 2022 and afforded serial number PCT/US2022/049067, which claimed, pursuant to 35 USC 119, priority to, and the benefit of, the earlier filing date of US Provisional Patent Applicant Ser. No. 63/276,592 filed on Nov. 6, 2021, the contents of which are incorporated by reference, herein.

### BACKGROUND OF THE INVENTION

### Field of the Invention

This invention relates to the field of medical examination/diagnosis and treatment and more particularly to a devices utilizing Fluorescent Enhancement Theragnosis technology in the identification and treatment of medical conditions related to the oral cavity.

### Background Information

Light based therapy has emerged as a useful tool in locating, identifying and treating infection or bacterial load within bodily tissues and reducing or eliminating the bacterial load within the tissues. Light based therapy is intended to provide for photoactivated disinfection, photodynamic therapy and photobiomodulation.

Photoactivated therapy is based on the use of inactive prodrugs whose biological activity is significantly increased upon exposure to light. See, for example, F. Reeßing, et al., Beyond Photodynamic Therapy: Photoactivated Cancer Chemotherapy, Curr. Med. Chem., 2018, 24, 4905-4950.

Photoactivated disinfection (PAD) is based on the concept of photodynamic therapy (PDT). PDT is a treatment method that is associated with the use of a light source and a photosensitizer (PS) that binds to cells and after absorption of light induces a chemical reaction that results in the release of radicals and singlet oxygen. See, for example, Husejnagic S, et al., Photoactivated disinfection in periodontal treatment: A randomized controlled clinical split-mouth trial. J Periodontol. 2019; 90:1260-1269.

Photobiomodulation (PBM) is a non-invasive method that contributes to pain relief and reduces inflammation, parallel to the enhanced healing and tissue repair processes. See, for example, Dompe, C., et al., M. Photobiomodulation-Underlying Mechanism and Clinical Applications. J. Clin. Med. 2020, 9, 1724.

The referred to research has found that the application of light within known wavelength ranges when applied to a surface is an effective means for reducing the activity of virus and bacteria. Such research, referred to, herein, as Fluorescent Enhancement Theragnosis (FET) technology, has proved itself to provide benefit to practitioners, such as dentist, doctors and surgeons, etc. in identifying bacteria that would not normally be viewable by the practitioner.

For example, FET has been found use in dental practice to apply light to a patient's mouth to identify virus and/or bacteria and to reduce the activity of virus/bacteria to limit the infection from the virus/bacteria.

Similarly, FET technology provides a doctor or surgeon the ability to view virus and/or bacteria on or within a patient and initiate appropriate remedial actions when necessary.

However, the light utilized in FET technology may be harmful to the human eye and, thus, eye protection is required for both the patient and the practitioner to avoid inadvertently viewing the harmful light. And further generates a significant amount of heat that prevents devices used in applying the FET technology to remain for extended periods of time within the patient.

Hence, there is a need in the industry for applying a light therapy to the oral cavity that provides for the detecting and healing of infection caused by excessive bacterial and/or viral load to a patient while preventing inadvertent viewing of the applied light and providing for the retention of a device within the oral cavity for extended periods of time.

### SUMMARY OF THE INVENTION

Disclosed is device to apply light therapy to infection such as bacteria and viruses within an oral cavity of a patient wherein the device is adjustable to provide a customized fit of the device to the user.

In accordance with the principles of the invention, a light based therapy device is disclosed to provide for fluorescence based identification and treatment of virus/bacterial within an oral cavity, wherein the light based therapy device comprises at least one mouthpiece comprising a plurality of lighting sources positioned on at least one of: a top surface, a bottom surface, at least one lateral surface between the top surface and the bottom surface and the back (or flat) surfaces of the at least one mouthpiece, a connector configured to provide an electrical energy to the plurality of lighting sources, a control board configured to control signals to cause the electrical energy to be provided to selected ones of the plurality of lighting sources and a bite element configured to provide an indication of placement the mouthpiece addition.

In accordance with another aspect a light based therapy device is disclosed to provide a light treatment to an oral cavity of a patient, wherein the light based therapy device comprises a rear mouthpiece, and a front mouthpiece that are positioned on opposite sides of the patient's teeth, a bite element positioned between the rear mouthpiece and the front mouthpiece wherein the patient engages the bite element to retain the light based therapy device in position, a connector configured to provide electrical energy to a plurality of selected ones of a plurality of lighting sources associated with the rear mouthpiece and the front mouthpiece, wherein at least one of the rear mouthpiece and the front mouthpiece is slidable with respect to the connector.

In accordance with another aspect a light based therapy device suitable for insertion into a oral cavity to provide light therapy is disclosed wherein a cooling system is incorporated within the device to maintain temperature that allows for the long term retention.

For a better understanding of exemplary embodiments and to show how the same may be carried into effect, reference is made to the accompanying drawings. It is stressed that the particulars shown are by way of example only and for purposes of illustrative discussion of the preferred embodiments of the present disclosure and are presented to clarify the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

### BRIEF DESCRIPTION OF THE DRAWINGS

The advantages, nature, and various additional features of the invention will appear more fully upon consideration of the illustrative embodiments described in detail in connection with the accompanying drawings, where like or similar reference numerals are used to identify like or similar elements throughout the drawings:

FIG. 1 illustrates a perspective view of a first exemplary embodiment of a light treatment device in accordance with the principles of the invention.

FIG. 2 illustrates a front view of the first exemplary embodiment of the light treatment device shown in FIG. 1.

FIG. 3 illustrates a top view of the first exemplary embodiment of the light treatment device shown in FIG. 1.

FIG. 4 illustrates a side view of the first exemplary embodiment of the light treatment device shown in FIG. 1.

FIG. 5 illustrates a perspective view of a second exemplary embodiment of a light treatment device in accordance with the principles of the invention.

FIG. 6 illustrates a back view of the second exemplary embodiment of the light treatment device shown in FIG. 5.

FIG. 7 illustrates a top view of the second exemplary embodiment of the light treatment device shown in FIG. 5

FIG. 8 illustrates a cut-away side view of the second exemplary embodiment of the light treatment device shown in FIG. 5.

FIG. 9 illustrates an exploded perspective view of a third exemplary embodiment of a light treatment device in accordance with the principles of the invention.

FIG. 10 illustrates an exploded perspective view of a cooling system shown in FIG. 9

FIG. 11 illustrates a top view of the light treatment device shown in FIG. 9.

FIG. 12 illustrates a prospective view of a light treatment device and cooling system in accordance with the principles of the invention.

It is to be understood that the figures, which are not drawn to scale, and descriptions of the present invention described herein have been simplified to illustrate the elements that are relevant for a clear understanding of the present invention, while eliminating, for purposes of clarity, many other elements. However, because these omitted elements are well-known in the art, and because they do not facilitate a better understanding of the present invention, a discussion of such elements are not provided herein. The disclosure, herein, is directed also to variations and modifications known to those skilled in the art.

### DETAILED DESCRIPTION OF THE INVENTION

As used herein, the terms “comprises”, “comprising”, “includes”, “including”, “has”, “having”, or any other variation thereof, are intended to cover non-exclusive inclusions. For example, a process, method, article or apparatus that comprises a list of elements is not necessarily limited to only those elements but may include other elements not expressly listed or inherent to such process, method, article, or apparatus. In addition, unless expressly stated to the contrary, the term “of” refers to an inclusive “or” and not to an exclusive “or”. For example, a condition A or B is satisfied by any one of the following: A is true (or present) and B is false (or not present); A is false (or not present) and B is true (or present); and both A and B are true (or present).

The terms “a” or “an” as used herein are to describe elements and components of the invention. This is done for convenience to the reader and to provide a general sense of the invention. The use of these terms in the description, herein, should be read and understood to include one or at least one. In addition, the singular also includes the plural unless indicated to the contrary. For example, reference to a composition containing “a compound” includes one or more compounds. As used in this specification and the appended claims, the term “or” is generally employed in its sense including “and/or” unless the content clearly dictates otherwise.

All numeric values are herein assumed to be modified by the term “about,” whether or not explicitly indicated. The term “about” generally refers to a range of numbers that one of skill in the art would consider equivalent to the recited value (i.e., having the same function or result). In any instances, the terms “about” may include numbers that are rounded (or lowered) to the nearest significant figure.

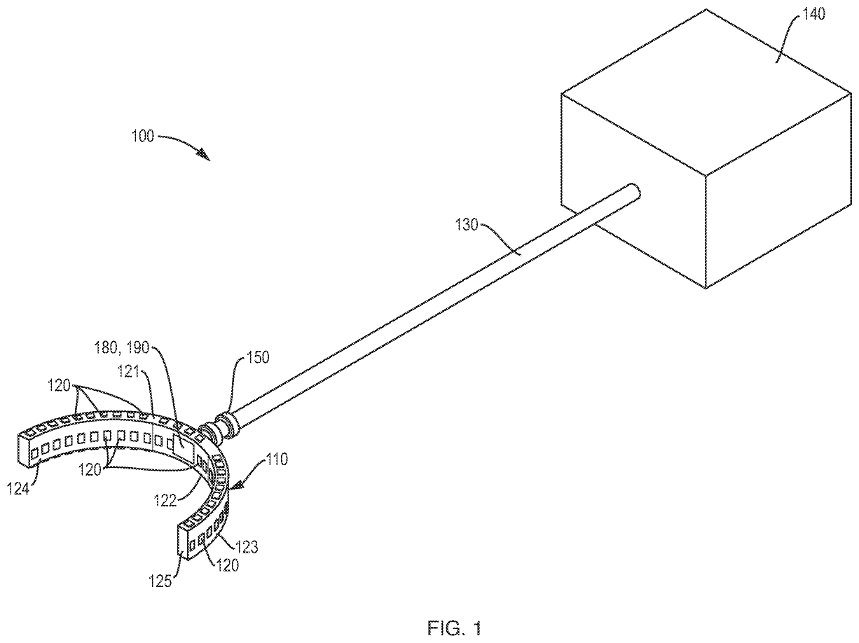

FIG. 1 illustrates a perspective view of a first exemplary embodiment of an oral cavity light-based therapy device 100 in accordance with the principles of the invention.

Light based therapy device 100 comprises a mouthpiece element 110 that may be formed, as illustrated, into a shape that is substantially conformal to an inner shape of a patient's mouth. Mouthpiece 110 may be composed of a flexible material (e.g., silicon) that enables mouthpiece 110 to conform to the surface of a patient's teeth, for example. Alternatively, mouthpiece 110 may be composed of a pliable or adaptable material that may be molded into a desired shape or configuration either when placed within the oral cavity or fitted against an inner surface of a patient's teeth.

In accordance with the principles of the invention, mouthpiece 110 further comprises a plurality of lighting sources (e.g., light emitting diodes) 120 positioned along at least one surface of mouthpiece 110, wherein the plurality of lighting sources 120 are positioned to emit light substantially perpendicular to the surface upon which the lighting sources or positioned. In the illustrated embodiment, lighting sources 120 are positioned on top surface 121, bottom surface 122, external lateral surface 123 and internal lateral surface 124 between top surface 121 and bottom surface 122. Although not shown, it would be recognized that lighting sources 120 may be positioned on back or flat end surfaces 125 to enable light to be directed to the back of the oral cavity.

The plurality of lighting sources 120 may be configured to emit light in one or more of a plurality of light wavelength bands. For example, an ultra-violet wavelength range (10-380 nanometers (nm)), a visible wavelength range, which may be one of white (i.e., 380-750 nm) or one or more of a color light (e.g., violet—380-435 nm; blue—435-495 nm; cyan—495-520; green—420-570 nm; yellow—570-590 nm; orange—590-620 nm and red—620-750 nm) and an infra-red wavelength range.

Although not shown in detail, it would be recognized by those skilled in the art that each of the plurality of lighting sources 120 may be composed of one or more light sources arranged in an exemplary configuration (e.g., matrix). For example, the illustrated lighting sources 120 may be composed of four (4) individual light sources (e.g., light emitting diodes), wherein one of the light sources may emit light in a white wavelength range, a second one of the light sources may emit light at a first wavelength in a blue wavelength range, a third one of the light sources may emit light at a second wavelength in the blue wavelength range and a fourth one of the light sources may emit light in an orange wavelength range. The light emitted by the selected one of the light sources within each of the lighting sources 120 may be emitted individually, concurrently and or sequentially.

Although lighting sources 120 are discussed with regard to a combination of four light sources, it would be recognized that the number of light sources per lighting source 120 may be increased and decreased without altering the scope of the invention. Similarly, the wavelength ranges of the light sources (i.e., the light emitted) within each of the lighting sources 120 may be altered from the exemplary wavelength ranges disclosed, herein, without altering the scope of the invention claimed.

In one aspect of the invention, mouthpiece 110 may comprise a heat-transferrable material (i.e., cooper, aluminum, etc.) that may be encased in a waterproofing material (e.g., silicon). Mouthpiece 110 may then be formed or molded into a desired shape to allow for proper positioning of mouthpiece 110 in the oral cavity. Light emitted by the lighting sources 120 may be transmitted through an optically clear material toward tissue within the oral cavity. Alternatively, the waterproofing material may be translucent or frosted to provide for a more diffuse output of the light emitted.

Lighting sources 120 may be positioned on printed circuits boards (not shown) that may be positioned on an encased heat transferable material (e.g., cooper, aluminum, etc.), within mouthpiece 110. The heat transferrable material within mouthpiece 110 may further provide for the transmission of electrical energy to lighting sources 120 while removing heat generated by lighting sources 120 from the oral cavity. Alternatively, lighting sources 120 may be positioned on a flexible printed circuit board (not shown) that may be formed or molded into a shape that provides for a desired placement of lighting sources 120 within the oral cavity.

Further illustrated is connection 130 that provides a path for electrical energy to be supplied from control module 140 to lighting sources 120. Control module 140 may comprise a power source or power supply (not shown) (i.e., a conventional battery (e.g., alkaline), a rechargeable battery and/or an AC/DC converter that converts alternating current to direct current). Connection 130 may further include a protective sheathing composed of a material that provides protection for the electrically elements within connector 130. Within connector 130 a heat conductive material may be included that provides a heat conductive path from which heat generated by the operation of lighting sources 120 may be drawn from the oral cavity.

Although control module 140 is shown remote from mouthpiece 110, and connected through connection 130, it would be recognized that connection 130 may be such that control module 140 is positioned substantially proximal to mouthpiece 110, as will be discussed.

In accordance with the principles of the invention, control module 140 may further include electrical elements (i.e., resistors, capacitors, integrated circuits, etc.) (not shown) positioned on a control board (i.e., printed circuit board) (not shown) that may be used to control an application of a voltage or current to selected ones of the plurality of lighting sources 120 on or within mouthpiece 110.

In one aspect of the invention, control module 140 may include a user interface that enables a user to manually provide input instructions for the application of a voltage or current, the time duration of the application of such voltage or current, the level of such voltage or current and/or the color or color temperature to selected ones of the plurality of lighting sources. The control module 140 may further determine whether the voltage or current are applied in a pulsed (e.g., pulse repetition rate, pulse duration, pulse on/off ratio (i.e., duty cycle) etc.) or a continuous manner. For example, Pulse Width Modulation (PWM) is a well-known pulse technique where the pulse duration (or width) is altered between pulses to provide a longer or a shorter “ON” time during the duration of the time a signal is to be active (i.e., pulse mode duration). In this example, longer or shorter times for a light to be emitted for the duration of the time light is to be emitted.

In an alternative aspect of the invention, control module 140 may include a plurality of pre-set light setting that are set based on a desired light-based treatment for a known condition.

Further illustrated is bite element 150 positioned proximal to mouthpiece 110 along connector 130. Bite element 150 provides a region upon which a patient may place their teeth to retain mouthpiece 110 in a proper relationship within the patient's oral cavity.

In one aspect of the invention, bite element 150, which may be made of a rubber or other similar material (e.g., silicon), is slidable along wire connection 130. The slideability of bite element 150 is advantageous as it enables the placement of a front surface of mouthpiece 110 to be substantially adjacent a patient's front teeth. For example, mouthpiece 110 may be adjusted within the oral cavity to be substantially adjacent the rear surfaces of a patient's teeth by providing a light pressure onto bite element 150 while sliding mouthpiece 110 into position. Alternatively, mouthpiece 110 may be placed deeper into the oral cavity to position light sources 120 closer to a point of infection.

In addition, bite element 150 may comprise a pressure sensor, a contact sensor, a proximity sensor, or other similar type sensing element (not shown) that may provide (or provides) an indication to control module 140 that bite element 150 has been engaged. Control module 140, receiving the provided indication of engagement of bite element 150 (and, hence, the proper positioning of mouthpiece 110), may provide (or provides) voltage to selected ones of the plurality of lighting sources 120. Without the indication of proper placement (i.e., engagement of a user's teeth) from bite element 150, control module 140 is inhibited from providing a voltage to the plurality of lighting sources 120.

In accordance with the principles of the invention, an image capture device (e.g., a camera, a CCD element, etc.) 180 may be incorporated onto (or within) interior lateral surface 124 to allow for the capturing of images or video of the oral cavity before, during and after treatment by the light-based therapy system 100.

Images captured by image capture device 180 may be transmitted, via connector 130, to a control board (not shown) within control module 140, wherein the images may be subsequently transmitted through a wireless or wired transmitter to a display device (not shown) or stored on a storage media (not shown). Protocols for wireless (e.g., BLUETOOTH, Wi-FI, etc.) and wired (e.g., RS-232) communications are well-known in the art and a detailed discussion of transmitting/receiving data utilizing these or similar (e.g., NFC) protocols is not believed necessary in discussing the invention disclosed, herein.

in accordance with the principles of the invention, a filter 190 may be placed before image capture device 180, wherein filter 190 limits the wavelength range captured by image capture device.

In one aspect of the invention, filter 190 may be configured to block the viewing or capturing of light wavelength ranges in a first wavelength, for example. In one aspect of the invention, filter 190 may be configured to block the wavelength region of light emitted by lighting sources 120, while allowing light associated with one or more specific or desired wavelength regions to be viewed or captured. In one aspect of the invention, filter 190 may comprise a notch filter configured to block wavelengths emitted by selected ones of said plurality of lighting sources 120 while allowing wavelengths outside of the notch filter range to pass through to image capture device 180. Alternatively, filter 190 may comprise a long pass filter that blocks light emitted by selected ones of said plurality of lighting sources 120 below a known wavelength while allowing wavelengths above the known wavelength to be viewed. As an example, lighting sources 120 may emit light in a blue light wavelength range and in a white wavelength range, wherein filter 190 may possess filter characteristics that form a notch filter to blocks the blue light from being captured by image capture device 180, while allowing the white light, excluding that wavelength region blocked by the notch filter, to be viewed by the image capture device 180.

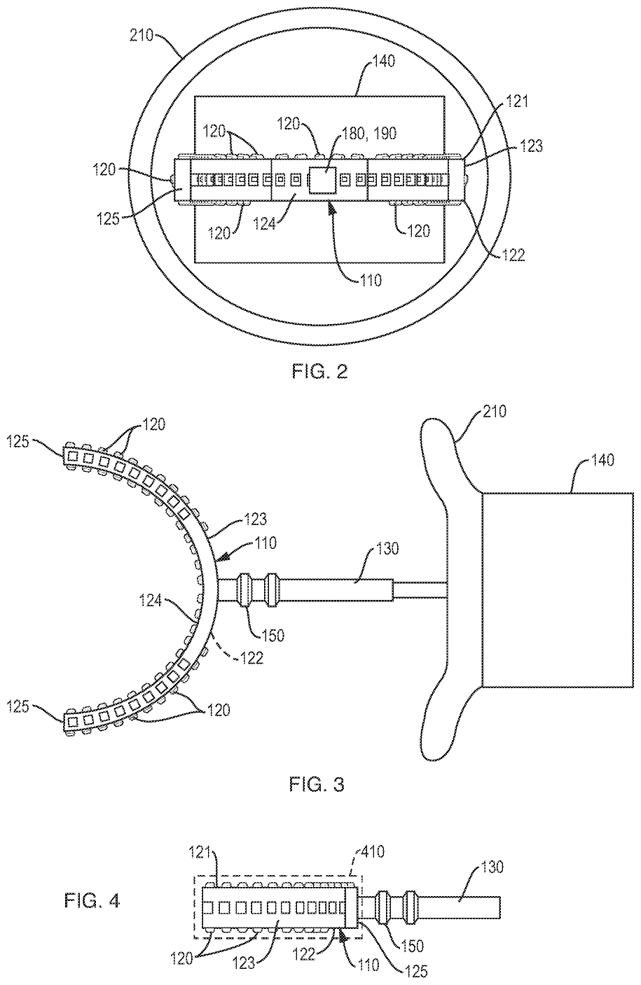

FIG. 2 illustrates a front view of oral cavity light-based therapy device 100 shown in FIG. 1, wherein the view is taken from the oral cavity outwardly toward device 100. In this illustrated embodiment is shown internal lateral surface 124 of mouthpiece 110 and control module 140, wherein lighting sources 120 are positioned on each of top surface 121, bottom surface 122, external lateral surface 123 and internal lateral surface 124 of mouthpiece 110.

Further illustrated is mask 210 and control module 140 wherein mask 210 blocks light emitted by lighting sources 120 from exiting the patient's mouth, when control module 140/mask 210 combination is pressed against the patient's mouth. In this aspect of the invention, mask 210 may comprise a flexible material that may be pressed against a patient's lower face (e.g., the mouth area) that is configured to block light emitted by lighting sources 120 from exiting the oral cavity.

Alternatively, mask element 210 may be a separate element that may be slidable with respect to connector 130 to allow the positioning of mask element 210 adjacent the lower face area of a patient after mouthpiece 110 is inserted into the oral cavity.

FIG. 3 illustrates a top view of light-based therapy device 100 shown in FIG. 1. In this illustrated embodiment is shown connector 130 and bite element 150, which is positioned proximal to a front portion 310 of external lateral surface 123. Further illustrated are lighting sources 120 positioned on a top surface 121, external lateral surface 123 and internal lateral surface 124 of mouthpiece 110. In one aspect of the invention, mask 210 may include a contact sensor, a proximity sensor, a heat sensor, etc., that indicates mask 210 is properly positioned with respect to a user. The sensor may provide an indication to the control module 140 that indicates the proper positioning of mask 210 with respect to the use or the patient. In this case, control module may be configured to provide electrical energy to selected ones of the lighting sources after receiving the indication of proper position of mask element 210.

FIG. 4 illustrates a side view of light-based therapy device 100 shown in FIG. 1, wherein lighting sources 120 are positioned on top surface 121, bottom surface 122 and external lateral surface 123. Although not shown, and as previously discussed, lighting sources 120 may be positioned on the end (back or flat) surface 125 positioned between top surface 121 and bottom surface 122.

Further illustrated is encasing material 410, which as discussed, may be composed of a waterproofing material (e.g., silicon) that prevents liquid from contacting the lighting sources 120, and the associated electrical components. Encasing material 410 further allows for the sterilization of the mouthpiece 110. In one aspect of the invention, encasing material 410 may be composed of an optically clear material. In another aspect of the invention encasing material 410 may comprise a translucent or frosted material. In this case, the light emitted by lighting sources 120 may be diffused as it passes through a translucent or frosted encasing material 410.

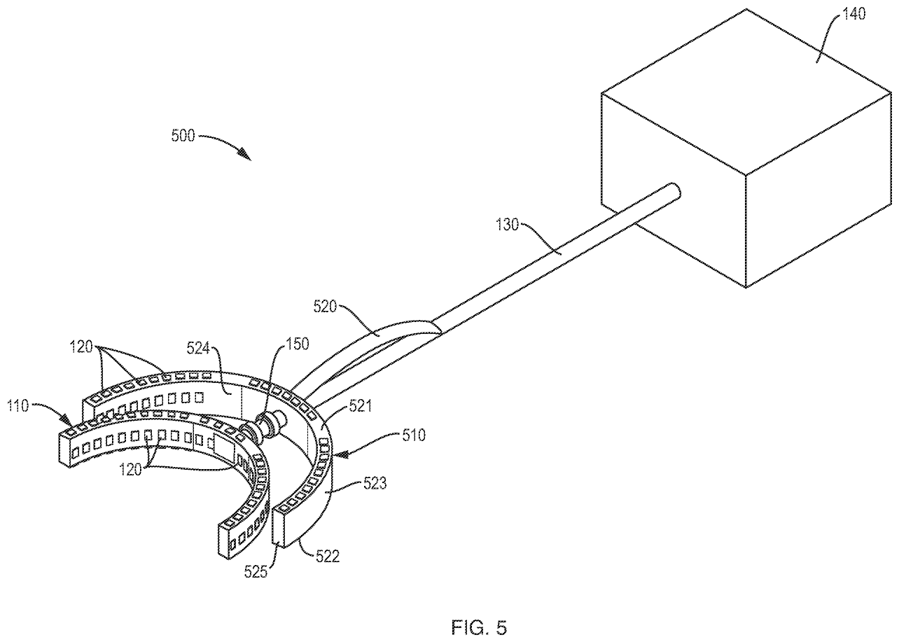

FIG. 5 illustrates a perspective view of a second exemplary embodiment of an oral cavity light-based therapy device 500, in accordance with the principles of the invention. In this illustrated second exemplary embodiment, light-based therapy device 500, similar to the light-based therapy device 100 shown in FIG. 1, comprises control module 140, connector 130, bite element 150 and mouthpiece 505, wherein mouthpiece 505 comprises mouthpiece 110, shown in FIG. 1 (referred to, hereinafter, as rear mouthpiece 110) and front mouthpiece 510. Front mouthpiece 510, similar to rear mouthpiece 110, comprises a plurality of lighting sources 120 arranged to emit light into an oral cavity.

In this illustrated embodiment, front mouthpiece 510 is positioned along connector 130 such that bite element 150 is positioned between rear mouthpiece 110 and front mouthpiece 510. Front mouthpiece 510, similar to rear mouthpiece 110, may be composed of a flexible or a pliable material that may conform substantially to a front portion of the oral cavity (e.g., the front surfaces of a patient's teeth). In one aspect of the invention front mouthpiece 510 may further include a heat-transferrable material similar to that described with regard to rear mouthpiece 110.

Front mouthpiece 510 further includes a plurality of lighting sources 120 arranged, in this example, on top surface 521, a bottom surface 522 and a rear surface 524 of front mouthpiece 510. Although not shown, it would be recognized that lighting sources 120 may be incorporated onto the back (or edge) surfaces 525 of front mouthpiece 510, as discussed with regard to rear mouth piece 110, without altering the scope of the invention claimed.

In one aspect of the invention, rear mouthpiece 110 and front mouthpiece 510 may be adjusted to substantially engage rear and front surfaces, respectively of a patient's teeth, for example, by the movement of rear mouthpiece 110 and/or front mouthpiece 510 with respect to bite element 150.

In accordance with the principles of the invention, rear mouthpiece 110 and front mouthpiece 510 may be positioned by the illustrated adjustment cable 520 to alter the position of one or both of rear mouthpiece 110 and front mouthpiece 510. For example, by adjusting the distance between rear mouthpiece 110 and front mouthpiece 510, rear mouthpiece 110 and front mouthpiece may be placed in a substantially adjacent relationship to the rear and front surfaces., respectively, of a patient's teeth. Alternatively, front mouthpiece 510 may be placed adjacent the front of the patient's teeth, while rear mouthpiece 110 may be extended toward a rear portion of the patient's oral cavity. Although an adjustment cable 520 is shown, to cause movement of at the illustrated elements of mouthpiece 505, it would be recognized that other forms of movement control may be incorporated into the illustrated second exemplary embodiment of a light therapy device. For example, rear mouthpiece 110 and front mouthpiece 510 may include a screw thread, which may be positioned on a screw that allows the adjustment of the position of one or both of rear mouthpiece 110 and front mouthpiece 510.

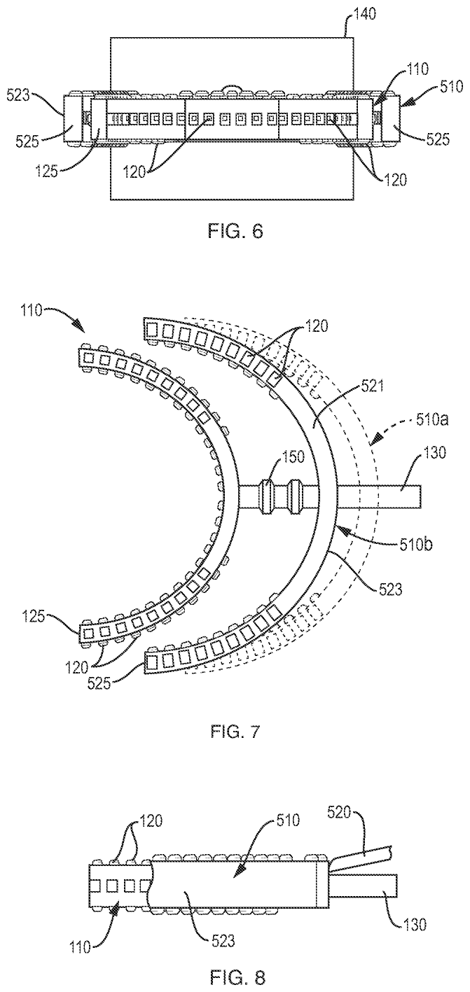

FIG. 6 illustrates a front view (when viewed looking outward from inside an oral cavity) of the second exemplary embodiment shown in FIG. 5. Similar to the configuration shown in FIG. 2, lighting sources 120 are shown on an internal lateral surface 124 of rear mouthpiece 110. Further shown is the external lateral surface 523 and back surface 525 of front mouthpiece 510.

FIG. 7 illustrates a top view of the second exemplary embodiment of the light-based therapy device shown in FIG. 5 showing the adjustable movement of front mouthpiece 510 with respect to bite element 150.

In this illustrated exemplary aspect of the invention, front mouthpiece 510 is shown in a first position or initial position (referred to as 510a) and then slide along connector 130 to a second position (referred to as 510b), which places mouthpiece 510 substantially adjacent a front surface of a patient's teeth (not shown). As shown, the shape of front mouthpiece 510 may be altered to conform to the front surface of the patient's teeth as front mouthpiece 510 is positioned substantially adjacent the patient's or user's teeth.

Light emitted by one or more lighting sources 120 positioned on top surface 521, bottom surface 522, interior surface 524 and flat surface 525 may emit light toward tissue (e.g., gums) at or near the front surfaces of a patient's teeth.

Although not shown in FIG. 7, it would be recognized that rear mouthpiece 110 may be similarly adjusted with respect to bite element 150, as discussed previously.

FIG. 8 illustrates cut-away side view of the second exemplary embodiment of mouthpiece 505 shown in FIG. 5 in accordance with the principles of the invention.

In this illustrated embodiment, outer surface 523 of front mouthpiece 510 fails to include lighting elements 120, whereas the outer surface 123 of mouthpiece 110 includes lighting elements 120 (FIG. 4). Although the outer lateral surface 523 of front mouthpiece 510 is shown to not include lighting elements 120, it would be in the knowledge of those skilled in the art to incorporate lighting elements 120 onto (or into) the outer lateral surface 523 of mouthpiece 510 to provide light therapy to the inner surfaces of the user's lips. Such modification of the configuration shown, herein, has been contemplated and considered by the inventors to be within the scope of the invention claimed.

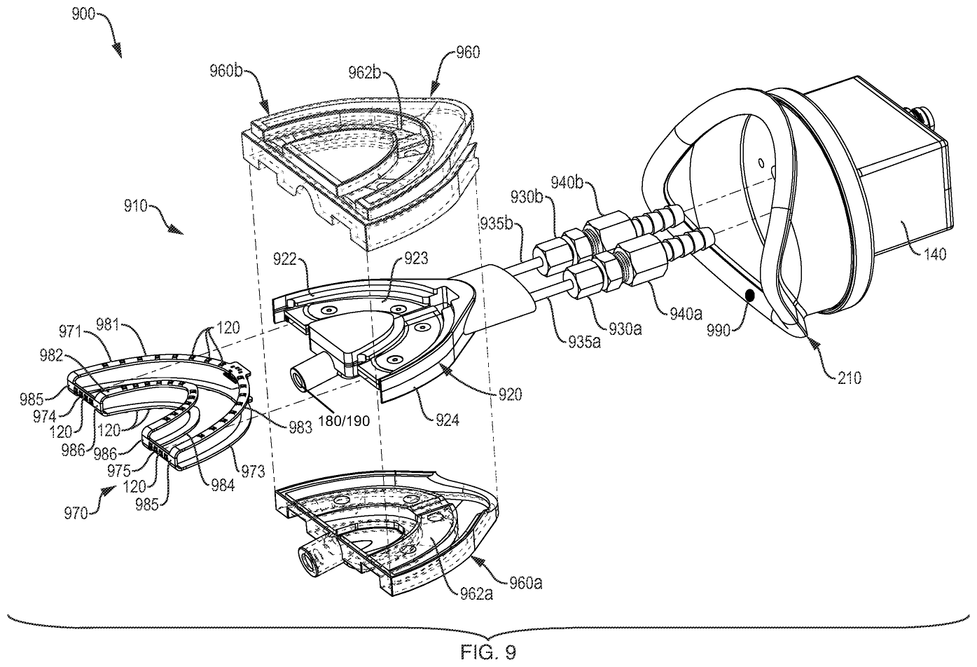

FIG. 9 illustrates an exploded perspective view of a third exemplary embodiment of a light treatment device in accordance with the principles of the invention.

In this exemplary embodiment, light treatment device 900 comprises a mouthpiece element 910 comprising a cooling plate 920, and a frame 970 comprising a plurality of lighting sources 120, wherein cooling plate 920 and the lighting structure 970 are contained within a protective element 960 (shown in two parts as 960a,960b). Further illustrated are cooling lines 935a, 935b, which, as will be discussed, provide a cooling fluid (e.g., air, water, glycol, etc.) to cooling plate 920. Further illustrated are fitting 930a, 930b attached to the free ends of cooling lines 935a, 945b. Fittings 930a, 930b and their connection to fittings 940a, 940b, respectively, provide for the removable attached of device 900 from a cooling source (not shown).

Similar to the embodiment of the invention shown in FIGS. 2 and 3, mask 210 may be associated with mouthpiece 910 to construct light therapy device 900. As previously discussed, mask 210 prevents light emitted by the lighting sources 120 from exiting a body cavity and may further include a contact (or proximate contact) switch 990 to indicate proper placement of mouthpiece 910. In one aspect of the invention, switch 990 may comprise a proximity or contact switch that provides an indication of proper placement of mouthpiece 910.

In addition, control module 140 may be incorporated onto mask 210, wherein control module 140 provides control signal and/or voltage to one or more of the lighting sources 120. For example, control module 140 may include power sources (e.g., batteries) that provide voltage (or power) to lighting devices 120. Alternatively, a remote power source (see FIG. 1), such as an AC/DC converter may provide voltage (or power) to lighting devices 120.

Further illustrated is light frame or light structure 970 comprising a first frame 971 and a second frame 973, where for further discussion are referred to as upper frame 971 and lower frame 973, respectively. However, it would be understood that the reference to first frame 971 and second frame 973 as being upper and lower may be reverses without altering the scope of the invention claimed. In this illustrated embodiment, upper frame 971 comprises outer frame 981 and inner frame 982 and lower frame 973 comprises outer frame 983 and inner frame 984. Each of frames 981-984 incorporate a plurality of lighting sources 120 arranged on at least one surface, wherein light emitted by lighting sources 120 is directed away from a corresponding one of frames 981-984. As shown, upper outer frame 981 and lower outer frame 983 are joined together at a distal end by connectors 985. Similarly, upper inner frame 982 and lower inner frame 984 are joined by connector 986.

Further illustrated are connectors 998 which join the upper frame 971 and lower 973 at corresponding distal ends. In one aspect of the invention, connectors 985 and 986 may include one or more lighting sources 120 to direct light into the oral cavity.

In one aspect of the invention, lighting structure 970 may be composed of a heat-transferrable material (i.e., copper, aluminum, etc.), which may be placed in direct contact (i.e., adjacent) with cooling plate 920 to provide for a significant transfer of heat away from lighting sources 120. Alternatively, lighting structure 970 may be incorporated into recesses or channels on exterior surfaces of cooling plate 970.

In one aspect of the invention, an upper module 922 of cooling plate 920 may include a groove or channel 923 into which light structure 970 may be inserted. The insertion of light structure 970 into groove or channel 923 allows for broad contact of frame 970 with cooling plate 920 to draw heat generated by lighting sources 120 away. Although not shown, it would be recognized that a similar groove or channel may be incorporated into a lower module 924 of cooling plate 920.

In accordance with the illustrated embodiment shown, cooling plate 920 and light structure 970 may be encased or enclosed within a covering material 960, which is represented as an upper covering material 960b and a lower covering material 960a. Covering element 960 (represented as two parts 960a and 960b when joined together) provides a moisture resistance covering that prevents liquids from contacting lighting sources 120 or cooling plate 920. In one aspect of the invention, covering material 960 may be composed of one of: an optical clear material, a translucent material and a frosted material, wherein the wavelengths emitted by the lightings sources 120 may be transmitted through covering material 960.

In one aspect of the invention covering material 960 may include one or more channels 962b that provide for a “bite” surface into which a patient may place their teeth. In addition, a similar “bite” surface 962a may be incorporated into lower covering material 962a.

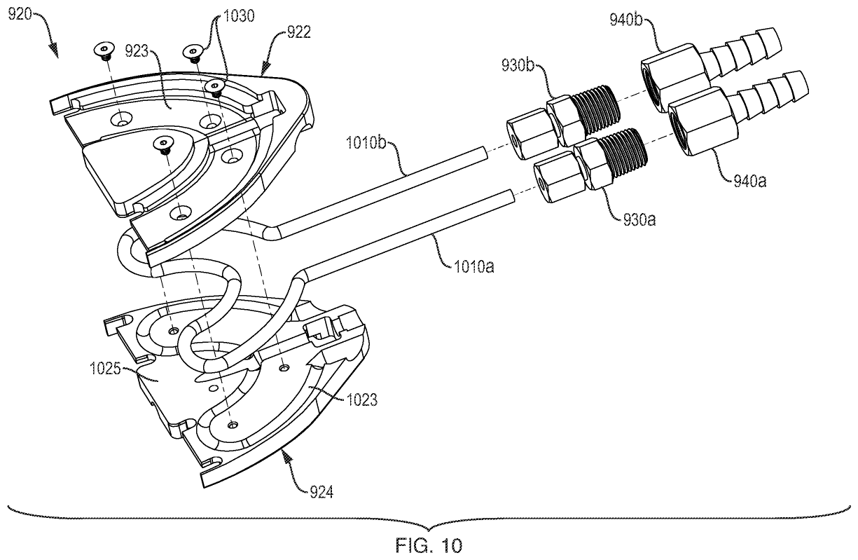

FIG. 10 illustrates an exploded perspective view of a cooling plate 920 shown in FIG. 9

As illustrated, cooling plate 920 be composed of a heat transferrable material, e.g., aluminum, cooper, etc., that includes upper module 922 and lower module 924, as previously discussed. Further shown in groove or channel 923 incorporated within upper module 922. In the aspect of the invention shown, upper module 922 and lower module 924 may be joined together through a screw connection, wherein screws 1030 may be inserted though pre-drilled holes in upper module 922 and lower module 924.

In accordance with the aspect of the invention shown, an inner surface 1025 of lower module 924 includes groove or channel 1023 into which may be placed cooling pipes 1010a, 1010b, wherein cooling pipes 1010a, 1010b are presented as a single entity, which provides a cooling fluid to cooling plate 920 to remove heat collected in the cooling fluid away from cooling plate 920. Although not shown, it would be recognized that inner surface (not shown) of upper module 922 may include a similar channel, wherein maximum contact between cooling plate 920 and cooling pipers 1010a, 1010b is achieved. Further shown is the removable attachment of fittings 930a, 930b from fittings 940a, 940b, respectively, which provide for the removable attachment of cooling plate 920 from a source of cooling fluid. Although a screw thread attachment is shown, it would be recognized and fittings 930a/940a and 930b/940b may be removably attachable through a compression connection, for example, or other similar connections that allows for a leak free connection.

Although cooling plate 920 is shown as a two-piece configuration, in another aspect of the invention, cooling plate 920 may be composed of a solid heat transferrable material wherein upper module 922 and lower module 924 are a single unit and cooling conduits or pipes 1010a, 1010b are represented as channels within cooling plate 920. Alternatively, conduits 1010a, 1010b may be positioned around cooling plate 920.

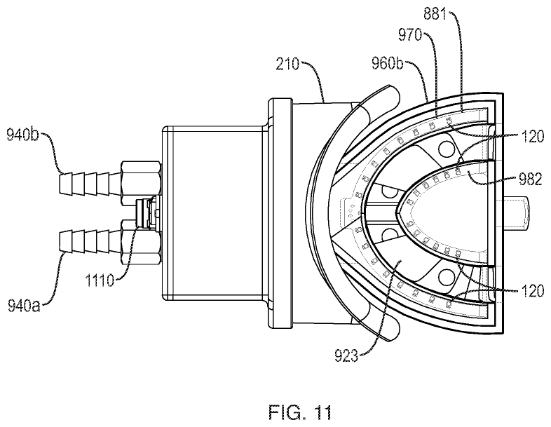

FIG. 11 illustrates a top view of the light treatment device shown in FIG. 9.

In this illustrated embodiment of light therapy device 900, lighting sources 120 are shown positioned on upper outer surface 981 and upper inner surface 982. Further illustrated is channel 923 positioned between upper outer surface 981 and upper inner surface 982. As would be appreciated, spacing of channel 923 may be determined based on an expected tooth size. For example, channel 923 may be sized for pediatric dental operation. Alternatively, the spacing of channel 923 may be sized to accommodate dental work on young adults or adults.

In one aspect of the invention, light therapy device 900 may be operated by a remote power source similar to that shown in FIG. 1, wherein connector 1110 provides for the electrical connection of the (not shown) power source with light therapy device 900.

In one aspect of the invention, one or more of channel 962a, 962b may include a proximity or contact switch that provides an indication of proper placement of mouthpiece 910.

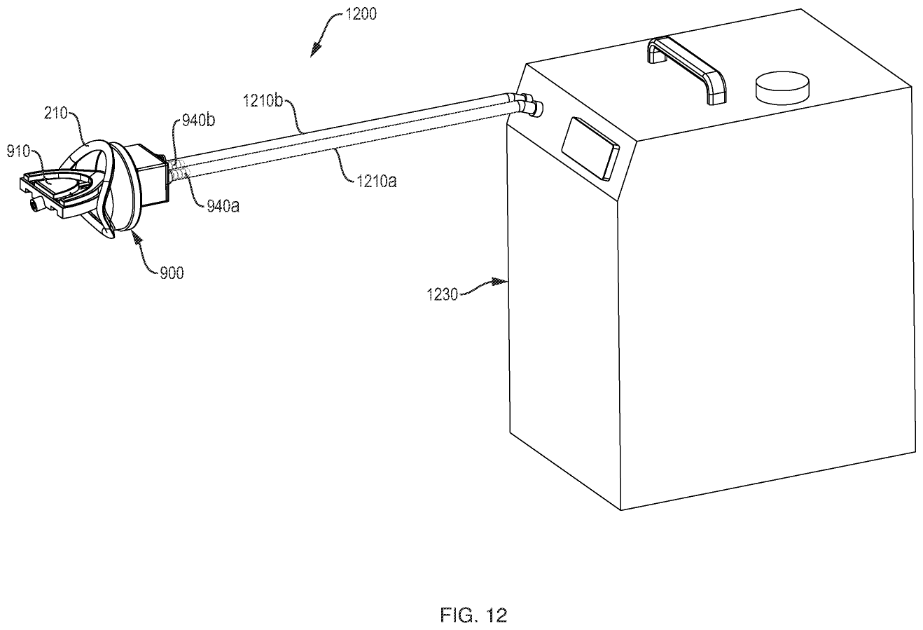

FIG. 12 illustrates a prospective view of a light treatment device and cooling system in accordance with the principles of the invention.

In this illustrated view of system 1200 comprising light therapy device 900 connected to a cooling system 1230, which is configured to provide cooling fluid (e.g., air, water, glycol, etc.) to light therapy device 900 through hoses 1210a, 1210b. Hoses 1210a and 1210b are connected to connectors 940a, 940b, respectively, which provides for the removable attachment of light therapy device 900 from cooling system 1230.

In one aspect of the invention, cooling element 1230 may include a temperature sensor, which monitors the returned heating cooling fluid temperature to determine the level of cooling achieved. In one aspect of the invention, the temperature of the cooling fluid provided to light therapy device 900 may be determined, in part, based on the temperature of the returned cooling fluid temperature. For example, the temperature of the provided cooling fluid may be decreased when the temperature of the returned fluid is greater than a desired value. Alternatively, the temperature of the cooling fluid may be increased when the temperature is below a desired value. Accordingly, the temperature of the light therapy device is maintained at a temperature that is compatible (i.e., not too hot, not too cold) with the body cavity. The maintenance of the temperature allows for a long-term insertion of the light therapy devices shown, herein, in the body cavity.

Although lighting structure 970 is shown as a fixed configuration, it would be recognized that the cooling plate 920 and associated cooling apparatus, shown in FIGS. 9 and 15, may be applicable to the lighting structure 100 and 100/510 shown in FIGS. 1 and 5, respectively. For example, lighting structure 100 or 100/510 may be placed on or within channels within a cooling plate (not shown) similar to that shown in FIG. 9.

In summary, a mouthpiece element has been disclosed that provides for the placement of light emitting elements within an oral cavity of a patient or a user wherein an adjustment means is used to customize the position of the mouthpiece element(s) with respect to the patient's or user's oral cavity. In one aspect of the invention, the mouthpiece element(s) may be positioned substantially adjacent to the teeth of the patient or the user. In another aspect of the invention, the mouthpiece element may be positioned extended into the oral cavity to provide light therapy surrounding or near the patient's or user's throat. In addition, a control mechanism is disclosed that provides for the safe application of a voltage to the light emitting elements within or on the surface of the mouthpiece element(s). In one aspect of the invention, a facial mask element may be incorporated into the mouthpiece element (110, 505 or 900) to reduce the possibility of the viewing of those wavelengths of the lighting elements that may be harmful to the eyes.

Although the invention disclosed herein discusses specific wavelengths that are produced with currently available LEDs (i.e., non-lasing light emitting diodes and laser diodes), it would be recognized that the specific wavelengths absorbed and/or reflected may be changed and/or added to without altering the scope of the invention. In addition, it would be known in the art that the specific wavelengths discussed, herein, represent a band of wavelengths centered on the wavelength values presented herein to account for divergence of the wavelength generated by the LED during the generation of the light and/or the operation of the LED, wherein the light generated is represented as a nominal value.

The invention has been described with reference to specific embodiments. One of ordinary skill in the art, however, appreciates that various modifications and changes can be made without departing from the scope of the invention as set forth in the claims. Accordingly, the specification is to be regarded in an illustrative manner, rather than with a restrictive view, and all such modifications are intended to be included within the scope of the invention.

Benefits, other advantages, and solutions to problems have been described above regarding specific embodiments. The benefits, advantages, and solutions to problems, and any element(s) that may cause any benefits, advantages, or solutions to occur or become more pronounced, are not to be construed as a critical, required, or an essential feature or element of any or all of the claims.

1. A light-based therapy device comprising:
a mouthpiece comprising:
a base plate, said base plate comprising:
at least one channel formed within an interior surface of said base plate; and
at least one channel formed within at least one exterior surface of said base plate;
a light structure in contact with said base plate, said light structure comprising:
a first frame comprising:
a first outer frame; and
a first inner frame, wherein said first outer frame and said first inner frame are positioned on opposite sides of said at least one channel in said exterior surface of said base plate, said exterior surface being an upper surface of said base plate, wherein said outer frame and said inner frame comprise:
a plurality of lighting sources configured to direct emitted light away from said base plate; and
a second frame, said second frame comprising:
a second outer frame; and
a second inner frame, wherein said second outer frame and said second inner frame are positioned on opposite sides of said at least one channel on said exterior surface of said base plate, wherein said exterior surface being a lower surface of said base plate, wherein said second outer frame and said second inner frame comprise:
a plurality of lighting sources configured to direct emitted light away from said base plate; and
a control module configured to:
receive electrical energy from a power source; and
provide said electrical energy to selected ones of said plurality of lighting sources associated with said first frame and said second frame.

2. The light-based therapy device of claim 1, comprising:
a mask element configured to:
slide with respect to said mouthpiece.

3. The light-based therapy device of claim 1, comprising:
a mask element attached to said control module.

4. The light-based therapy device of claim 1 comprising:
a covering material comprising a material selected from a group consisting of: a flexible material and a pliable material, wherein said covering material is configured to:
encase said mouthpiece, wherein said covering material is selected from a group consisting of: optically clear, translucent and frosted.

5. The light-based therapy device of claim 1, comprising:
a conduit positioned within said at least one channel in said interior surface of said base plate, said conduit configured to:
circulate a cooling fluid within said base plate.

6. The light-based therapy device of claim 1, comprising:
a conduit positioned about said base plate, said conduit configured to:
circulate a cooling fluid to said base plate.

7. The light-based therapy device of claim 1, wherein said base plate comprises a heat-transferrable material.

8. The light-based therapy device of claim 1, wherein said plurality of lighting sources emit light in at least one of a plurality of light wavelength bands, wherein said light is emitted individually, concurrently or sequentially.

9. (canceled)

10. (canceled)

11. The light-based therapy device of claim 1, wherein said first frame and said second frame are joined together at a distal end of said frame element.

12. The light-based therapy device of claim 1, comprising:
an image capture device, wherein images captured by said image capture device are provided to said control module.

13. The light-based therapy device of claim 12 comprising:
a filter configured to:
limit a wavelength range of light captured by said image capture device to a desired wavelength range.

14. The light-based therapy device of claim 1, wherein said electrical energy provided by said control module is provided in one of: a pulse mode and a continuous mode, wherein characteristics of said pulse mode is at least one of: pulse duration, pulse repetition rate, pulse duration, and a pulse on/off ratio.

15. A lighted mouth piece device comprising:
a base element, comprising:
a lower section comprising:
an external facing lower surface, said external facing lower surface comprising:
a lower surface channel therein; and
an upper section comprising:
an external facing upper surface, said external facing upper surface comprising:
an upper surface channel therein; and
a lighted frame element comprising:
an upper frame, said upper frame comprising:
an outer upper frame; and
an inner upper frame, each of said outer upper frame and said inner upper frame comprising a plurality of lighting elements configured to emit a light, wherein said upper frame is positioned within said upper surface channel; and
a lower frame comprising:
an outer lower frame; and
an inner lower frame, each of said outer lower frame and said inner lower frame comprising a plurality of lighting elements configured to emit a light, wherein said upper frame and said lower frame are joined at a distal end of said upper frame and said lower frame, and
a conduit positioned within said base element, wherein said conduit is configured to:
circulate a cooling fluid within said at least one channel within said base plate; and
a control module configured to:
receive electrical energy from a power source; and
provide said electrical energy to selected ones of said plurality of lighting elements.

16. The mouthpiece of claim 15 further comprising:
an upper cover material configured to encase said upper section; and
a lower cover material configured to encase said lower section, wherein said upper cover material is configured to extend into said upper channel within said upper section between said upper outer frame and said upper inner frame and said lower cover material is configured to extend into said lower channel between said lower outer frame and said lower inner frame.

17. The mouthpiece of claim 16, wherein said upper covering material and said lower covering material comprising a material selected from a group consisting of: a flexible material and a pliable material.

18. The mouthpiece of claim 15, wherein said upper covering material and said lower covering material being selected from a group consisting of: optically clear, translucent and frosted.
